(19) United States
(12) Patent Application Publication
(10) Pub. No.: US 2021/0188773 A1
(43) Pub. Date: Jun. 24, 2021

(54) NOVEL POLYMOPHS OF S-NITROSOCAPTOPRIL (CAP-NO) AND ITS PROCESS FOR PREPARATION

(71) Applicant: FUZHOU UNIVERSITY, Fuzhou, Fujian (CN)

(72) Inventors: LEE JIA, BOYDS (US); YUYANG ZHOU, FUZHOU CITY, FUJIAN PROVINCE (CN); FAN CHEN, FUZHOU CITY, FUJIAN PROVINCE (CN); FEIYANG LI, FUZHOU CITY, FUJIAN PROVINCE (CN); MIN LIN, FUZHOU CITY, FUJIAN PROVINCE (CN); SHUHUI LI, FUZHOU CITY, FUJIAN PROVINCE (CN)

(21) Appl. No.: 16/761,284

(22) PCT Filed: Mar. 20, 2018
(86) PCT No.: PCT/CN2018/079619

§ 371 Date: May 4, 2020

(30) Foreign Application Priority Data
Jan. 25, 2018 (CN) 201810070716.7

(51) Int. Cl. C07D 207/16

(52) U.S. Cl. CPC C07D 207/16; C07B 2200/13

(57) ABSTRACT

Disclosed herein is a novel monohydrate polymorphic form of S-nitrosocaptopril (CapNO), process for preparation, pharmaceutical compositions and method of treating pulmonary hypertension, hypertension, or congestive heart failure thereof. A process for the preparation of S-nitrosocaptopril crystalline form is provided which comprises the following steps: reacting captopril, sodium nitrite and EDTA-2Na.2H₂O in 15±5 wt % saline, adjusting pH to precipitate the crystal, and recrystallizing S-nitrosocaptopril monohydrate crystalline. The crystals can be stably stored at 4° C. for at least 12 months.

### FIELD OF THE DISCLOSURE

The present invention provides novel polymorphic form of S-nitrosocaptopril, process for preparation, pharmaceutical compositions and method of treating pulmonary hypertension, hypertension, and congestive heart failure thereof.

### BACKGROUND OF THE DISCLOSURE

Captopril, known as Capoten (Cap), is the first orally effective angiotensin-converting enzyme inhibitor (ACEI) (Jia Li. Angiotensin-converting enzyme inhibitor Progress in Research. Progress in Physiological Sciences, 1985, 16: 229-234). In 1977, Ondetti et al completed the chemical synthesis and preliminary pharmacological experiments of Cap. Over the next two decades, ACEI is widely used in the treatment of hypertension and heart failure all over the world. Since the 1990s, with patent expired, Chinese generic Caps has been launching in a large scale in order to meet the needs of 1-2.7 billion hypertensive patients, prompting China immediately to be the world's largest Cap producer. However, novel ACEIs such as Enalapril and Lisinonpril have been gradually replacing Cap. Therefore, to optimize and modify the existing Cap production technology plays a leading innovational role in expanding the Chinese Cap industry to produce new Cap-derived drugs with high efficacy and few side effects.

Clinical data of ACEI, especially Cap, provide a wealth of valuable references in developed countries such as the United States. After oral administration of Cap, some of it is excreted in the form of urine, but most of the others are mainly metabolized into dimercapto conjugates (Cap-Cap), which likewise own the ability of hypotensive effect. It is well-known that when taking in a large amount of ACEI, acute renal failure is a common adverse reaction, or more especially, about 1.2% proteinuria, 25% nephrotic syndrome, 10% taste changes, drug eruptions, fever, itching, and eosinophilia in the early stages. When Cap long-term exposing, 5-20% of patients may suffer from dry cough, sometimes accompanied by wheezing and a few myelosuppression. However, the adverse reactions rarely occur in ACEI without reducing group-sulfhydryl(—SH) (e.g. Enalapril, Lisinopril, etc.). In that case, whether the adverse reactions can be overcome via replacing original reducing thiol (—SH) of Cap and transforming to nitroso (—NO) whose hydrolysate, nitric oxide, dilate the renal artery and bronchus simultaneously.

S-nitrosocaptopril (CapNO) was produced under the guidance (compound patent) which can be traced back to 1988-1989. It is based on Cap and transformed the thiols group (—SH) to nitrosyl thiol group (—SNO). Thus, the new compound, S-nitrosocaptopril (CapNO), possesses dual functions containing both NO releasing and ACEI in vivo. According to the biochemical reaction formula of the RSNO compound, the decomposition products are RSSR (such as Cap-Cap, Cap-SR) and NO whose end products are NO2 and NO3.

CapNO is a new mechanism drug targeting to SGC (soluble guanylate). The dominant pharmacological effects include 1. The ACEI effect of the parent compound, captopril which continuously dilates vessels thereby reducing pulmonary hypertension; 2. The role of NO which 1) directly resists to the formed vasoconstrictor substances; 2) selectively reduces pulmonary vascular resistance, pulmonary artery pressure thereby mildly relaxing respiratory smooth muscle to lower respiratory resistance, increasing pulmonary circulation blood flow and improving lung ventilation/blood flow ratio; 3) inhibits platelet aggregation; 4) mildly inhibits gastrointestinal smooth muscle; 5) expands vessels in vitro or in vivo for hypertension models; 6) increases tumor blood flow and oxygen consumption thereby enhancing tumor sensibility to radiotherapy; 7) reduces intraocular pressure and increases ocular fluid outflow which is beneficial to the treatment of glaucoma.

The main advantages of Cap-NO compared to Cap are: 1) It can slightly inhibit gastrointestinal secretion and movement. Therefore, Cap-NO is suitable for patients who suffer from hypertension associated with gastric ulcer. Because, most of the patients appear the symptom of a comparatively parasympathetic nerve advantage, such as gastric bowel is secreted increase, cankerous disease aggravates, etc. after taking sympathetic nerve inhibitors. Cap-NO is the very potential drug that may overcome the side effects. 2) Relaxing the respiratory smooth muscle which is an extra therapeutic effect compared with Cap, thereby probably applying in the treatment of occlusive emphysema and chronic cardiac insufficiency caused by pulmonary congestion, in other words, reducing respiratory resistance, increasing pulmonary circulation blood flow, and improving lung Ventilation/blood flow ratio. On the other hand, the patients may avoid dry cough and wheeze inducing by Cap. 3) Preventing atherosclerosis. Unlike Cap-NO, Cap has yet found any effect on preventing atherosclerosis; 4) Lowering blood cholesterol. 5) Unlike Cap, Cap-NO is an effective drug for both oral administration and injection. 6) Our recent results indicate that Cap-NO significantly inhibits the process of tumor cells adhesion to vascular endothelial cells, suggesting that Cap-NO may play a role in preventing tumor metastasis.

The prominent drawback of Cap-NO is the unstable nitrosyl hydrazide group (—SNO) whose decomposition free energy is only about 30 kcal/mol, which is prone to homogenization and release of NO. In fact, this is a necessary step for Cap-NO exerting its efficacy, but the chief culprit of instability. As a result, the property becomes a double-edged effect. Although easily decomposing in vitro, it is difficult to industrialize. If this technical bottleneck cannot be solved, even if Cap-NO's efficacy is superior, it will be difficult to be marketized.

We have studied in depth the nitrosyl hydrazide based on the chemical structure and found that —SNO is a special group, in fact presenting multiple properties containing partial double bonds, and ion pair. When the ion pair property is dominant group e.g. high temperature, metal ion, alkaline condition or strong acidity, the S—N single bond tends to break. Thus, the compound hydrolyzes in a short period. Only when the —SNO is in a specific chemical condition can the S-nitrosocaptopril be stabilized. Thereafter, the decomposition slowed down. Therefore, it is especially necessary to prepare a solid form of S-nitrosocaptopril, or especially a stable crystalline.

We are the only team who are capable to synthesize the Cap-NO monohydrate crystals. Meanwhile, researches related to Cap-NO were performed by a liquid form in other laboratories all over the world considering the challenge of Cap-NO crystal synthesis. But the present invention, somehow, lays the foundation of Cap-NO marketization and pharmaceuticalization. Our former method (preparation and quality testing of S-nitrosocaptopril crystals, patent No. CN1072208) to obtain Cap-NO was to mix equimolar Cap with NaNO2 in an aqueous solution thereafter adjusting the pH to ≤2.5 and stirring. And finally, S-nitrosocaptopril crystallized as an organic acid through draining and drying.

The present invention provides a novel process of Cap-NO.H2O preparation to avoid the hydrolysis of the S—N single bond by 1) reducing the probability of proton (H⁺) nucleophilic attack towards S atom during reaction; 2) H₂O and S—N bond interaction, specifically, hydrogen bond and dipole-dipole interaction; 3) adding EDTA-2Na.2H₂O in advance to complex catalytic ion e.g. Cu²⁺, Cu⁺, Fe²⁺ and Fe³⁺ and the like. Based on wherein optimized preparation, a monohydrate S-nitrosocaptopril can be obtained at a higher pH and resist higher temperature for a longer period. However, it can gradually decompose and release NO, and exert the drug effect after administration with crystal water losing.

### SUMMARY OF THE DISCLOSURE

In one aspect, this was the first attempt of applying this technology to synthesize S-nitrosocaptopril in the shape of more stable forms, considering the fatal drawback, instability. The present invention of monohydrate Cap-NO was discovered occasionally which was able to be reserved at 4° C. for more than 12 months.

Cap-NO.H2O, S-Nitrosocaptopril monohydrate, is L-Proline, 1-[2-methyl-3-(nitrosothio)-1-oxopropyl]-, (S)-, S-nitrosocaptopril monohydrate crystal, whose formula is showed bellow:

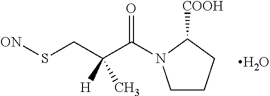

In another aspect, the present invention provides processes for the preparation of the crystalline form of S-nitrosocaptopril, which owns properties of simplicity, repeatability, and productivity.

In another aspect, the present invention provides a preparation method of pharmaceutical compositions for pulmonary hypertension, hypertension, and congestive heart failure.

In another aspect, the present invention provides a therapeutic method for pulmonary hypertension, hypertension and congestive heart-failure by pharmaceutical compositions comprising the polymorphic forms of S-nitrosocaptopril.

In another aspect, the present invention incorporates techniques showed below: Captopril, sodium nitrite and EDTA-2Na.2H₂O are resolve in saline solution following the pH adjusting to obtain crystal precipitation of S-nitrosocaptopril monohydrate.

Preferably, wherein the processes comprising:

a) Dissolving captopril (200˜300 parts by weight), sodium nitrite (70˜100 parts by weight) and EDTA-2Na.2H₂O (0.4˜1.5 parts by weight) in 0° C. precooled saline solution (700˜1000 parts by weight) and starred for about 15 to 120 minutes;
b) Adjusting the pH to 4 by hydrochloric acid (70˜100 parts by weight) for S-nitrosocaptopril monohydrate crystallization;
c) Filtering and draining the red mixture to obtain a crystal of S-nitrosocaptopril monohydrate.

Step-(a) should be carried out below 10° C.

The EDTA-2Na.2H₂O in Step-(a) should be added before the reaction.

The saline solution in Step-(a) is 15±5 wt %.

The hydrochloric acid in Step-(b) is 12 wt %˜24 wt %. Preferably, the hydrochloric acid in Step-(b) is 18 wt %˜24 wt %.

The pH in Step-(b) is about 4.0

Step-(c) should be carried out below 40° C.

Necessarily, the sodium nitrite in Step-(a) should be slightly more excess than that of captopril for complete reaction.

In another aspect, the purpose of adding EDTA-2Na.2H₂O in Step-(a) is to complex the metal ions such as Cu²⁺, Cu⁺, Fe²⁺ and Fe³⁺ potentially existing in the reaction system to avoid the S—N single bond of S-nitrosocaptopril being catalytically hydrolyzed.

In another embodiment, the content of the saline solution in Step-(a) is 15±5 wt % which increases the precipitation pH by a common-ion effect. Thus, unlike in strong acid solution, S atoms in S—N single bond can be largely protected as much as possible by lowering the opportunity of being attacked by protons (H⁺).

In another aspect, the rotation speed in Step-(a) should not be lower than 600 rpm, otherwise, S-nitrosocaptopril monohydrate crystals probably agglomerate when precipitated.

In another aspect, the present invention provides pharmaceutical compositions comprising the polymorphic forms of S-nitrosocaptopril prepared according to the processes of the present invention in any of its combination drug formulae in treatment of pulmonary hypertension, hypertension, and congestive heart failure.

In yet another aspect, the present invention further encompasses a process for preparing a pharmaceutical co-formula comprising combining any one of the polymorphic forms of S-nitrosocaptopril prepared according to processes of the present invention in any of its embodiments, with one or more pharmaceutically acceptable carrier in treatment of pulmonary hypertension, hypertension, and congestive heart failure.

In another aspect, wherein “a pharmaceutically acceptable carrier” means a conventional pharmaceutical carrier in the pharmaceutical field, including diluents or excipients such as water, starch, pregelatinized starch, sucrose, etc., binders such as a cellulose derivative, lactose, gelatin, polyvinylpyrrolidone Wetting agents such as glycerin, etc.; disintegrating agents such as agar, calcium carbonate, sodium hydrogen carbonate, etc.; absorption enhancers such as quaternary ammonium salts; surfactants such as cetyl alcohol, etc.; adsorption carriers such as kaolin; lubricants such as talc, magnesium stearate, polyethylene glycol, etc.; and other excipients such as flavoring agents and the like.

In another aspect, such pharmaceutical compositions may be administered to a mammalian patient in any dosage which may be adapted for administration to the patient by oral, nasal inhalation, rectal or parenteral administration or any other acceptable route of administration. Oral dosage forms include, but are not limited to, tablets, coated tablets, powders, granules, capsules and the like. Rectal or parenteral administration include, but are not limited to, suppositories, ointments, suspensions and the like. Preferably the dosage forms are tablets, coated tablets, granules, capsules and the like, and more preferred forms are coated tablets and capsules.

In another aspect, the present invention encompasses various dosage forms of the pharmaceutical compositions which can be prepared according to conventionally pharmaceutical methods. For example, the S-nitrosocaptopril monohydrate crystals can be mixed with one or more carriers for desired dosage forms.

In another aspect, the present invention encompasses 0.1 to 50 wt % or, preferably, 0.5 to 30 wt % S-nitrosocaptopril monohydrate crystals.

In another aspect, the beneficial effect of the present invention is showed below. The present invention encompasses a stable crystalline form, S-nitrosocaptopril monohydrate crystal, which can be stored at a relatively higher temperature for a long time. It could be prepared for various administration forms with properties of simplicity, repeatability, and productivity. The crystals were characterized by TG-DSC, PXRD, UV-vis, FTIR, Raman, etc., which proved to be a novel discovery. The S-nitrosocaptopril monohydrate crystals were applied in the treatment of hypertensive and pulmonary hypertension rats, presenting positive effects.

### BRIEF DESCRIPTION OF THE DRAWINGS

FIG. 1. shows the X-ray powder diffraction pattern of S-nitrosocaptopril monohydrate crystals;

FIG. 2. shows the DSC pattern of S-nitrosocaptopril monohydrate crystals;

FIG. 3. shows the Fourier transform infrared spectroscopy (FTIR) pattern of S-nitrosocaptopril monohydrate crystals;

FIG. 4. shows the Raman spectroscopy pattern of S-nitrosocaptopril monohydrate crystals;

FIG. 5. shows the UV-vis spectrum and its Partial enlargement of S-nitrosocaptopril monohydrate crystals;

FIG. 6. shows the Experimental diagram of the stability of S-nitrosocaptopril monohydrate crystals; and

FIG. 7. shows the micrograph of S-nitrosocaptopril monohydrate crystals.

### DETAILED DESCRIPTION OF THE EXEMPLARY EMBODIMENTS

The present invention will be further elaborated below in conjunction with the embodiments to enable those skilled in the art to more fully understand the present invention. Any person skilled in the art within the technical scope disclosed by the present invention may make changes without creative efforts or Replacement should be covered within the protection scope of the present invention. Therefore, the protection scope of the present invention should be subject to the protection scope defined by the claims.

### Example 1: Preparation of S-Nitrosocaptopril Monohydrate Crystals

Captopril (10 g), sodium nitrite (3.5 g) and EDTA-2Na.2H₂O (20 mg) were dissolved in 0° C. precooled 15% w/w saline (35 mL). Mixture stirring at 700 rpm for 1 h in ice bath, HCl (24% w/w) was added dropwise until pH reduced to 4.0. The resulted solid was filtered and dried at room temperature (25° C.). The resulted solid should be stored in refrigerator at 4° C. or −20° C.

### Example 2: Preparation of S-Nitrosocaptopril Monohydrate Crystals

Captopril (10 g) were sodium nitrite (4 g) were dissolved in 0° C. precooled 15% w/w saline (35 mL). Mixture stirring at 1000 rpm for 15 min in ice bath, HCl (18% w/w) was added dropwise until pH reduced to 4.0. The resulted solid was filtered and dried in an oven not exceeding 40° C. for 1 to 8 hours. The resulted solid should be stored in refrigerator at 4° C. or −20° C.

### Example 3: X-Ray Diffraction Pattern of S-Nitrosocaptopril Monohydrate Crystals

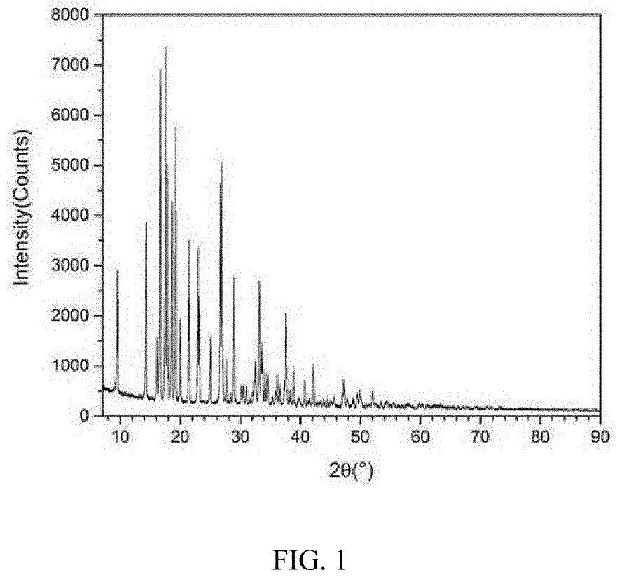

The powder X-ray diffraction pattern of S-nitrosocaptopril monohydrate crystal obtained according to the method of EXAMPLE 1 were substantially in accordance with FIG. 1 and PXRD pattern was showed in the following table:

| 2θ | D value of interplanar spacing | relative strength |
|---|---|---|
| 9.529 | 9.2741 | 33.1 |
| 14.309 | 6.1846 | 49.2 |
| 16.133 | 5.4892 | 17.5 |
| 16.659 | 5.3173 | 93.2 |
| 17.499 | 5.0638 | 100 |
| 17.828 | 4.9712 | 66.6 |
| 18.603 | 4.7657 | 57.3 |
| 19.233 | 4.6111 | 80.5 |
| 19.955 | 4.4457 | 22.5 |
| 21.491 | 4.1313 | 47.4 |
| 22.936 | 3.8742 | 44.3 |
| 23.184 | 3.8334 | 30.2 |
| 24.998 | 3.5592 | 19.4 |
| 26.638 | 3.3437 | 67.1 |
| 26.915 | 3.3099 | 73.3 |
| 28.910 | 3.0858 | 38 |
| 33.099 | 2.7042 | 39 |
| 33.479 | 2.6744 | 17.6 |
| 33.703 | 2.6571 | 16.5 |
| 37.576 | 2.3916 | 29.7 |

### Example 4: The Simultaneous TG/DSC Curves of S-Nitrosocaptopril Monohydrate Crystals

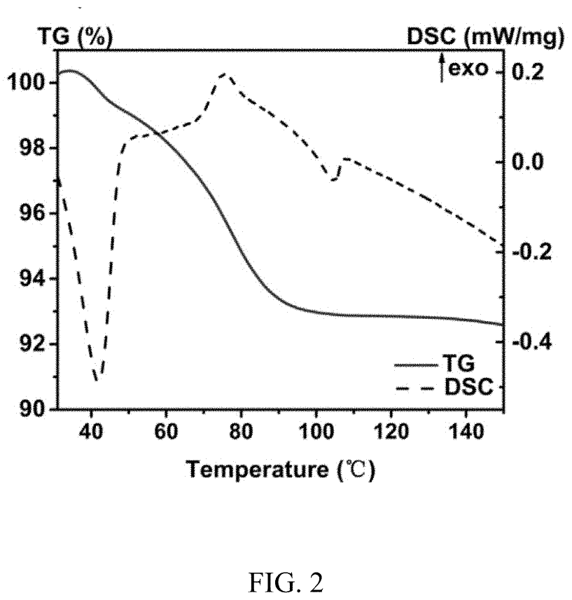

The S-nitrosocaptopril monohydrate crystal obtained according to the method of EXAMPLE 1 were heated at a rate of 2.0° C./min over a range of 25-150° C. in a N2 stream (FIG. 2). when the temperature reached 41.9° C., and at that point and thereafter, CapNO.H₂O lost its weight rapidly until 104.0° C., where there was a small endothermic peak in the DSC curve. The phase change of CapNO.H₂O from its solid state to oil state over the temperature range from 41.9 to 104° C. but the unique red color did not fade away, suggesting that CapNO.H₂O loses its water molecule without decomposition in S—NO bond of CapNO. Its relative weight loss value was 7.1%, which is very close to its theoretical water content of CapNO.H₂O, i.e., 6.8%, indicating a stoichiometric CapNO:H₂O ratio 1:1. On the other hand, if the NO group is lost from CapNO, the latter's relative weight loss should be 11.4%.

### Example 5: The FT-IR and Raman Spectra of S-Nitrosocaptopril Monohydrate Crystals

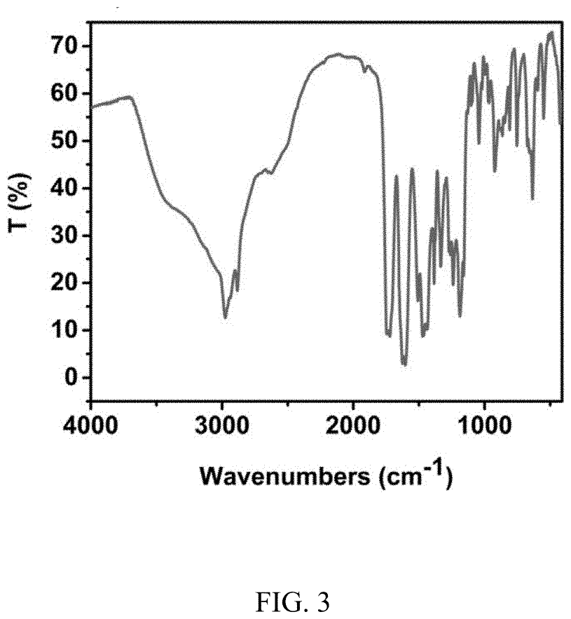

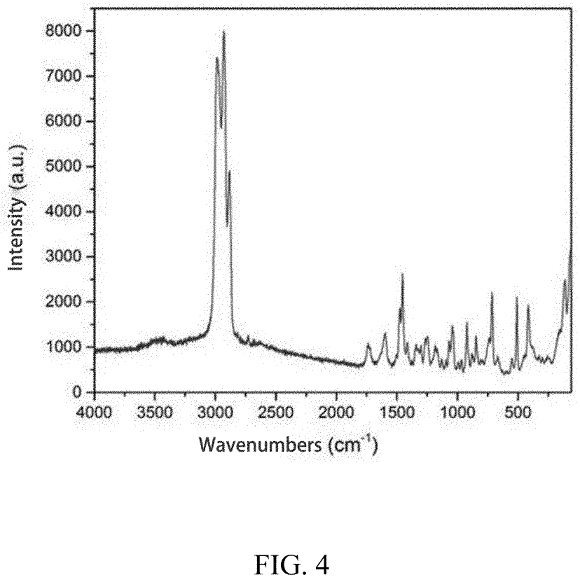

The S-nitrosocaptopril monohydrate crystals obtained according to the method of EXAMPLE 1 were further characterized by a Fourier infrared (FT-IR) and Raman spectra (FIG. 3 and FIG. 4). FT-IR showed absorption bands at 1334 cm-1, 1507 cm⁻¹ (N═O), 1102 cm⁻¹ (S—N), 2975 cm⁻¹ (ν O—H) 1625 cm⁻¹ (δ H₂O);

and Raman showed the strength bands at 2988 cm⁻¹, 2930 cm⁻¹ and 2882 cm⁻¹ which furthermore made sure of O—H bond existing.

### Example 6: The UV-Vis Spectra of S-Nitrosocaptopril Monohydrate Crystals

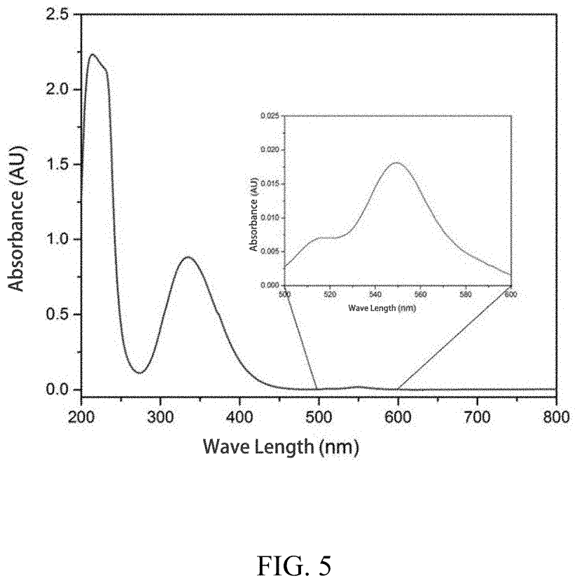

The S-nitrosocaptopril monohydrate crystals obtained according to the method of EXAMPLE 1 were further characterized by UV-vis spectra. UV-vis spectra of CapNO.H2O methanol solution (2 mg/mL) were recorded over a wavelength range of 800-200 nm at room temperature. The result showed that the maximum absorption at 333 and 55 him (FIG. 5), which are consistent with the UV-vis characteristics of CapNO.H2O crystals.

### Example 7: S-Nitrosocaptopril Monohydrate Crystals Stability Experiment

The S-nitrosocaptopril monohydrate crystals obtained according to the method of EXAMPLE 1 were divided into two parts, placed in petri dishes, spread into thin layers less than 5 mm and reserved at a) 4° C.±2° C., relative humidity 60%+10% or b) 25° C.±2° C., relative humidity 60%±10% respectively for 3 months (90 days). During the experiment, the first 60 days, every 5 days; 60-90 days, every 10 days were sampled once to inspect visually for the physical appearance and dissolve into methanol (2 mg/mL) for contents analysis of CapNO.H₂O by the HPLC whose column temperature was maintained at 40° C. with isocratic elution of methanol-phosphoric acid solution (pH=2) (50/50, v/v) at the flow rate of 1.0 mL/min and the UV-vis detection wavelength were 215 nm and 333 nm.

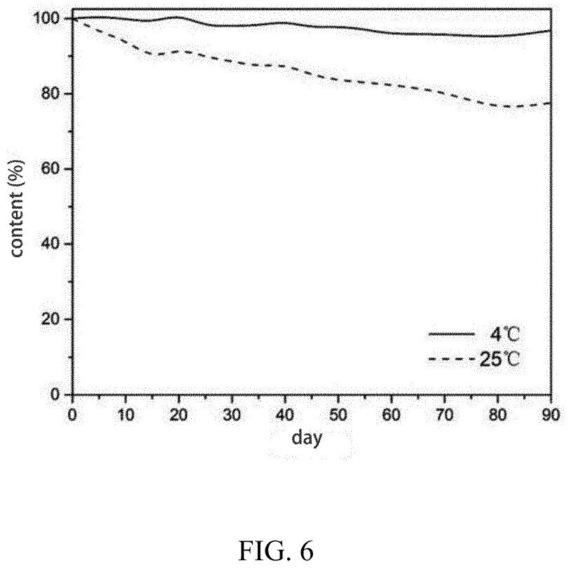

As is showed in FIG. 6, under the conditions of 4° C.±2° C. and relative humidity of 60%±10%, the contents only reduced by about 4.2% after 90 days, while at 25° C.±2° C., relative humidity of 60%±10% by about 22.4% after 90 days, indicating that the S-nitrosocaptopril monohydrate crystals of the present invention are very stable under the conditions of 4° C.±2° C. and relative humidity of 60%±10%.

### Example 8: The Micrograph of S-Nitrosocaptopril Monohydrate Crystals

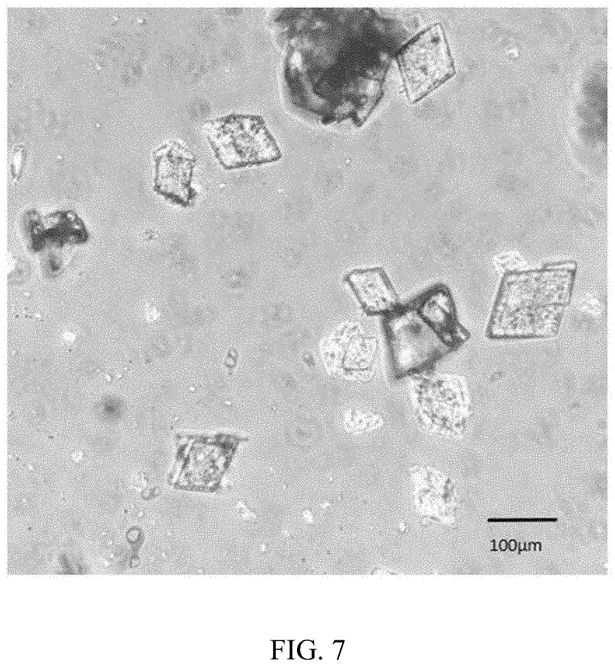

The S-nitrosocaptopril monohydrate crystals obtained according to the method of EXAMPLE 1 were examined under an optical microscope (magnification ×101), and found to be a rhombohedral crystal (FIG. 7).

### Example 9: The 20 g S-Nitrosocaptopril Monohydrate Tablet Properties

| Component | Percentage (%) | Weight (g) |
|---|---|---|
| Microcrystalline cellulose | 35.0 | 7.0 |
| Lactose monohydrate | 27.5 | 5.5 |
| Pregelatinized starch | 5.0 | 1.0 |
| corn starch | 5.0 | 1.0 |
| Stearic acid | 0.25 | 0.05 |
| S-nitrosocaptopril monohydrate | 27.25 | 5.45 |

S-nitrosocaptopril monohydrate crystals and the auxiliary materials preceding claimed were uniformly mixed, sieved and directly compressed by commonly tableting machines. The process produced at least 100 pieces of tablets and each of them weighed 200 mg, comprising 55.5 mg of S-nitrosocaptopril monohydrate crystals.

### Example 10: The 20 g Capsule of S-Nitrosocaptopril Monohydrate

| Component | Percentage (%) | Weight (g) |
|---|---|---|
| Lactose monohydrate | 27.5 | 14.8 |
| Magnesium stearate | 0.25 | 0.2 |
| S-nitrosocaptopril monohydrate | 27.25 | 5.0 |

S-nitrosocaptopril monohydrate crystals and excipients preceding claimed were uniformly mixed, sieved and directly filled into 100 gelatin capsules. Each of them weighed 200 mg, comprising 50 mg of S-nitrosocaptopril monohydrate crystals.

### Example 11: The Effect of S-Nitrosocaptopril Monohydrate Crystals on Hypertensive Model Rats

Two kinks of hypertensive rats including SHR (spontaneous hypertension model) and SS/Jr (salt-sensitive hypertension model) were randomly divided into three groups respectively: 0, 50 and 500 mg/kg doses of S-nitrosocaptopril monohydrate. Each group consisted of 10 female and 10 male rats. Rats of 50 mg/kg dose group voluntarily took in 5 mL freshly prepared S-nitrosocaptopril monohydrate water solution (10 mg/mL) (SHR hypertensive rats), or S-nitrosocaptopril 0.9% NaCl solution (SS/Jr hypertensive rat) every morning. Rats of 500 mg/kg dose group were administered orally once a day and measured weight once a week for 3 months. After 8 weeks, for SHR rats, 50 mg/kg dose group (n=7) revealed that weight, heart rate and systolic/diastolic blood pressure were 355±6 g, 356±75 beats/min, 126±10/111±23 mmHg, respectively, while the control group (n=7) revealed 350±4 g, 397±48 times/min, and 172±8/165±3 mmHg, respectively. The results showed that S-nitrosocaptopril had a significant antihypertensive effect (P<0.01). After 9 weeks, for SS/Jr rats, 50 mg/kg dose group (n=8) revealed that weight, heart rate and systolic/diastolic blood pressure were 349±8 g, 400±45 times/min, 123±20/109±28 mmHg, respectively, while the control group (n=8) revealed 379±9 g, 418±52 times/min, and 177±28/159±24 mmHg, respectively. The results showed that in SS/Jr rats, S-nitrosocaptopril had a significant antihypertensive effect (P<0.05). The comparative trials also showed that, blood pressure either SHR or SS/Jr rats was able to recovery to as equal as healthy Wistar rats (123±11/101±12 mmHg, n=7) by oral S-nitrosocaptopril treatment.

### Example 12: The Effect of S-Nitrosocaptopril Monohydrate Crystals on Pulmonary Hypertension Model Rats

Pulmonary hypertension (PAH) rats, being monocrotaline (MCT) intraperitoneal injected, were divided into 4 groups: control group (n=7), 50 mg/kg captopril group (n=7), 10 mg/kg S-nitrosocaptopril group (n=7), 50 mg/kg S-nitrosocaptopril group (n=7) and health group (n=9). Rats of all groups were orally administered once a day for 30 days. Results showed that there was no death in the healthy group. On the contrast, the number of rats in the control group dropped from 9 to 1. While the number of rats in 50 mg/kg captopril group and 10 mg/kg S-nitrosocaptopril group reduced from 7 to 3, the number of rats in 50 mg/kg S-nitrosocaptopril group was from 7 to 5. Dead rats were dissected in order to measure their average pleural effusion, which was disclosed to be 3.3 mL, 1.2 mL and 0.5 mL respectively in 50 mg/kg captopril group, 10 mg/kg S-nitrosocaptopril group and 50 mg/kg S-nitrosocaptopril group. In terms of mean right ventricular mass index (RVMI), control group, 50 mg/kg captopril group, 10 mg/kg S-nitrosocaptopril group, 50 mg/kg S-nitrosocaptopril group appeared to be 71.3%, 53.8%, 59.2%, 37.8%, respectively. The results indicated that S-nitrosocaptopril monohydrate can significantly improve the survival rate of PAH rats, lowered pulmonary artery pressure (P<0.05), reduced pleural effusion (P<0.05), and downregulated the right heart burden (P<0.05). Therefore S-nitrosocaptopril monohydrate has a significant therapeutic effect on pulmonary hypertension rats.

1. A location information displaying module, configured to display the location data that are resulted from conversion and are marked along the roads. A monohydrate S-nitrosocaptopril crystalline polymorph, wherein said monohydrate S-nitrosocaptopril is a monohydrate crystalline form of L-Proline, 1-[2-methyl-3-(nitrosothio)-1-oxopropyl]-, (S)-, comprising its structural formula embedded image

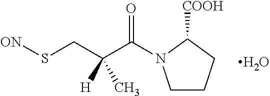

2. The crystalline form of S-nitrosocaptopril as claimed in claim 1 has at least one, or more, of the following characteristics: i) a powder X-ray diffraction pattern substantially in accordance with FIG. 1; ii) a powder X-ray diffraction pattern having peaks at about 9.529, 14.309, 16.133, 16.659, 17.499, 17.828, 18.603, 19.233, 19.955, 21.491, 22.936, 23.184, 24.998, 26.638, 26.915, 28.910, 33.099, 33.479, 33.703 and 37.576±0.2 degrees 2-theta substantially as depicted in FIG. 1.

3. A process for the preparation of S-nitrosocaptopril crystalline form of claim 1, comprising the following steps of
a) blending and reacting components in parts by weight: 200-300 parts of captopril, 70-100 parts of sodium nitrite, 0.4-1.5 arts of EDTA-2Na.2H2O and 700-1000 parts of 0° C. precooled saline solution for 15-120 minutes;
b) adding 60-90 parts of hydrochloric acid by pH=4.0;
c) straining and drying in vacuum to obtain the monohydrate S-nitrosocaptopril.

4. The process of claim 3, wherein step-(a) is reacted at a temperature below 10° C.

5. The process of claim 3, wherein the EDTA-2Na.2H₂O in step-(a) should be added before generating S-nitrosocaptopril.

6. The process of claim 3, wherein the sodium nitrite in step-(a) has NaCl consent of 15±5% by weight.

7. The process of claim 3, wherein the hydrochloric acid in step-(b) has HCl consent of 18-24% by weight.

8. The process of claim 3, wherein the straining and drying in step-(c) should be maintained at a temperature below 40° C.

9. A method for the treatment of one or more disorders selected from pulmonary hypertension, hypertension or congestive heart failure comprising administering a compound of claim 1 or a pharmaceutically acceptable salt thereof in a therapeutically effective amount to a patient in need thereof.

10. A pharmaceutical composition comprising the monohydrate S-nitrosocaptopril crystalline as claimed in claim 9 or a pharmaceutically acceptable salt thereof and pharmaceutically acceptable excipients.

11. A process for the preparation of S-nitrosocaptopril crystalline form of claim 2, comprising the following steps of
a) blending and reacting components in parts by weight: 200-300 parts of captopril, 70-100 parts of sodium nitrite, 0.4-1.5 arts of EDTA-2Na.2H₂O and 700-1000 parts of 0° C. precooled saline solution for 15-120 minutes;
b) adding 60-90 parts of hydrochloric acid by pH=4.0;
c) straining and drying in vacuum to obtain the monohydrate S-nitrosocaptopril.

12. The process of claim 9, wherein step-(a) is reacted at a temperature below 10° C.

13. The process of claim 9, wherein the EDTA-2Na.2H₂O in step-(a) should be added before generating S-nitrosocaptopril.

14. The process of claim 9, wherein the sodium nitrite in step-(a) has NaCl consent of 15±5% by weight.

15. The process of claim 9, wherein the hydrochloric acid in step-(b) has HCl consent of 18-24% by weight.

16. The process of claim 9, wherein the straining and drying in step-(c) should be maintained at a temperature below 40° C.

17. A method for the treatment of one or more disorders selected from pulmonary hypertension, hypertension or congestive heart failure comprising administering a compound of claim 2 or a pharmaceutically acceptable salt thereof in a therapeutically effective amount to a patient in need thereof.

18. A pharmaceutical composition comprising the monohydrate S-nitrosocaptopril crystalline as claimed in claim 17 or a pharmaceutically acceptable salt thereof and pharmaceutically acceptable excipients.
